(19) United States
(12) Plant Patent Application Publication
(10) Pub. No.: US 2002/0162148 P1
(43) Pub. Date: Oct. 31, 2002

(54) 'KRISTIN LIPKA'S VARIEGATED WEEPING DOGWOOD TREE'

(76) Inventor: Robert E. Lipka, Cedars, PA (US)

Correspondence Address: ROBERT E. LIPKA, R.E.L. NURSERY, CEDAR FARM, P.O. Box 106, CEDARS, PA, 19423, US

(21) Appl. No.: 09/845,701

(22) Filed: Apr. 30, 2001

(51) Int. Cl. A01H 5/00

(52) U.S. Cl. PLT/220

(57) ABSTRACT

A new and distinct Kousa Dogwood plant named Kristin Lipka's Variegated Weeper is illustrated and described.

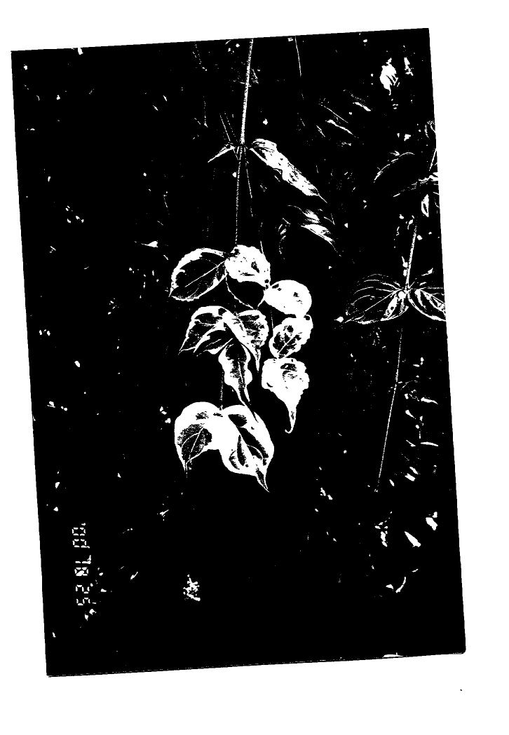

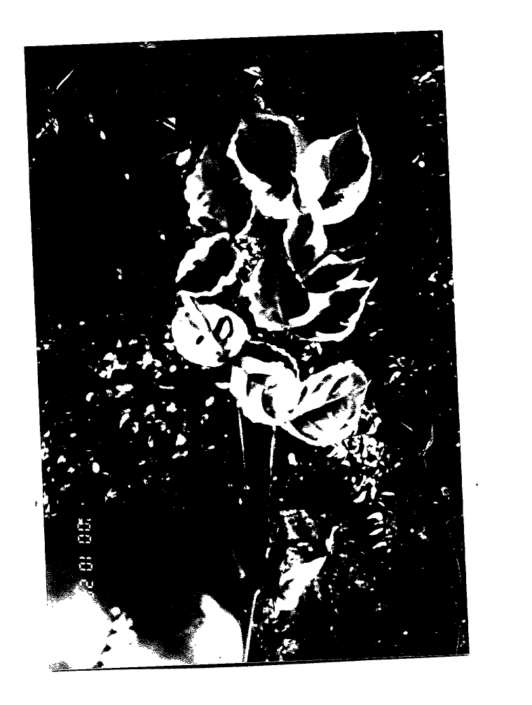

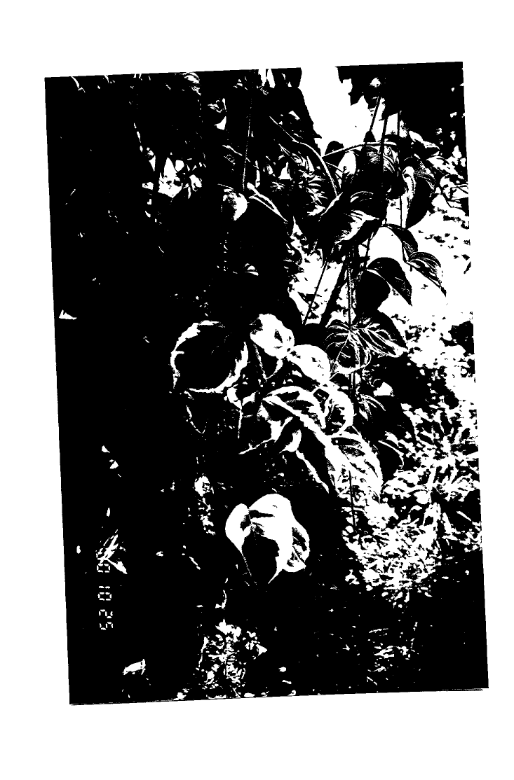

### BACKGROUND OF THE INVENTION

The present invention relates to a new and distinct cultivar of Kousa Dogwood, botanically known as Cornus Kousa, and hereinafter referred to by the cultivar name Kristin Lipka's Variegated Weeper.

The new Kousa Dogwood was discovered by the Inventor in a cultivated area in Cedars, Pa., as a naturally occurring branch sport of a plant of an unnamed variety of Cornus Kousa Pendula. This unnamed variety is described as having solid green (nonvariegated) leaves. The selection of this branch sport was based on its unique variegated foliage.

Asexual reproduction of the new Weeping Kousa Dogwood by grafting, has shown that the unique features of this new Weeping Kousa Dogwood are stable and reproduced true to type in successive generations.

### BRIEF SUMMARY OF THE INVENTION

Plants of the new Kousa Dogwood have not been observed under all possible environmental conditions. The phenotype may vary somewhat with variations in environment such as temperature, day length, light intensity, nutrition and water status without, however, any variance in genotype.

### DETAILED BOTANICAL DESCRIPTION

The following observations, measurements, values, and comparisons describe plants grown in Cedars, Pa., under outdoor conditions, which closely approximate commercial production conditions. Plants used for this description were about three years old and grown in quart containers.

Botanical classification: Cornus Kousa cultivar Kristin Lipka's Variegated Weeper.

Parentage: Naturally occurring branch sport of unnamed variety of Cornus Kousa described as having solid green (nonvariegated) leaves.

### PROPAGATION

Type: By grafting scions on to Cornus Kousa seedlings (understock). Rooting ability has not been determined at this date due to limited plant material.

The following traits have been repeatedly observed and are determined to be the unique characteristics of Kristin Lipka's Variegated Weeper. The characteristics in combination distinguish Kristin Lipka's Variegated Weeper as a new and distinct cultivar.

1. Medium to vigorous growth rate during early stages, slowing at maturity.

2. Large leaves with unique green and white variegated foliage.

The new Weeping Dogwood differs from the parent variety of Cornus Kousa Pendula primarily in leaf coloration as plants of the new Weeping Kousa Dogwood have variegated leaves whereas plants of the parent variety have nonvariegated, solid green leaves.

As of this date and to my knowledge, there are no known weeping varieties of a variegated Kousa Dogwood for comparison.

### BRIEF DESCRIPTION OF PHOTOGRAPHS

The accompanying colored photographs illustrate the overall appearance of the new weeping variegated Kousa Dogwood, showing the colors as true as it is reasonably possible to obtain in colored reproduction of this type.

The photograph on the first sheet comprises a close-up view of the weeping variegated Kousa Dogwood.

The accompanying photographs show the difference between the new variegated weeping mutation and the commonplace weeping variety on the same plant. Foliage colors in the photographs may appear different from the actual colors due to light reflectance.

Grafting is done in January using dormant scions on actively growing seedling understock.

Plant Description

Plant form and growth habit.—Perennial deciduous tree, with strong weeping habit.

Vigor.—Vigorous, similar to plants of the parent variety.

Plant size.—All plants grafted on 24 to 36″ Kousa understock in January and grown under outdoor Southeastern Pennsylvania conditions. Height and width unknown at this time, but it is assumed that the new cultivar will achieve height and width of a nonvariegated weeping variety.

Branching habit.—Strongly weeping.

Main stem, or trunk description.—Like species.

Bark texture.—Smooth, bark color, tan gray.

Foliage Description

Leaves.—Opposite, simple, elliptical-ovate, 2 to 4″ long, ¾ to ¾ wide, acuminate, cuneate center of leaf dark green with white irregular border. Occasional white splashes may randomly appear in the green center of the leaf area. Variegation patterns will be expressed on both surfaces of the leaves. In the fall a pinkish red hue becomes evident on the outer variegated area and becomes deeper pink with colder temperatures.

Bud.—Flower formed at end of stem, similar to species.

Stem.—Slender, light tan with tinges of purple and green, essentially glabrous.

Bark.—Exfoliation with age and forming a mottled mosaic of gray, tan and rich brown.

Size.—Similar to species.

Hardiness.—Zone 5 to 8.

Habit.—Pendulous and cascading.

Rate.—Medium to fast in early stages of growth.

Flowers.—Similar to species.

Fruit.—Drupe, pinkish red to red, ¾ to 1″ in diameter.

Diseases and Insects.—None serious, typical of species.

I claim:

1. A new and distinct Kousa Dogwood plant named Kristin Lipka's Variegated Weeper, as illustrated and described.
